(19) United States
(12) Patent Application Publication
(10) Pub. No.: US 2024/0350050 A1
(43) Pub. Date: Oct. 24, 2024

(54) SYSTEM COMPRISING INTEGRATED DICHOPTIC FLASH AND PUPILLOMETRY MONITORING, AND METHOD FOR USING THE SAME

(71) Applicant: Diagnosys LLC, Lowell, MA (US)

(72) Inventors: Bruce Doran, Westford, MA (US); Jeffrey Farmer, Chelmsford, MA (US); Kamyar Ghandi, Lowell, MA (US); Richard Robson, Holt (GB); Marc Chabot, Hollis, NH (US); Brian McLeod, Lowell, MA (US)

(21) Appl. No.: 18/684,933

(22) PCT Filed: Aug. 18, 2022
(86) PCT No.: PCT/US22/40719

§ 371 Date: Feb. 20, 2024

Related U.S. Application Data
(60) Provisional application No. 63/234,462, filed on Aug. 18, 2021.

(51) Int. Cl. A61B 5/16; A61B 3/11; A61B 3/14; A61B 5/11; A61B 5/297; A61B 5/398

(52) U.S. Cl. CPC A61B 5/163; A61B 3/112; A61B 3/14; A61B 5/1103; A61B 5/297; A61B 5/398

(57) ABSTRACT

Apparatus for obtaining ophthalmic electrophysiological, pupillometry and psychophysical responses from a test subject, the apparatus comprising: a housing comprising a curved surface; a dichoptic stimulator mounted to the curved surface of the housing, the dichoptic stimulator comprising a left eye stimulator for delivering a visual stimulus to the left eye of the test subject and a right eye stimulator for delivering a visual stimulus to the right eye of the test subject, whereby to evoke an electrophysiological response in the test subject; a pair of cameras mounted to the curved surface of the housing, wherein the pair of cameras comprises a left eye camera for obtaining images of the left eye and a right eye camera for obtaining images of the right eye of the test subject; and an indicator box for recording a psychophysical response to at least one of the visual stimulus to the left eye and the visual stimulus to the right eye.

### REFERENCE TO PENDING PRIOR PATENT APPLICATIONS

This patent application claims benefit of pending prior U.S. Provisional Patent Application Ser. No. 63/234,462, filed Aug. 18, 2021 by Diagnosys LLC and Bruce Doran et al. for INTEGRATED DICHOPTIC FLASH AND PUPILLOMETRY SYSTEM (Attorney's Docket No. DIAGNOSYS-15 PROV).

The above-identified patent application is hereby incorporated herein by reference.

### FIELD OF THE INVENTION

This invention relates to ophthalmic diagnostic equipment in general, and more particularly to a novel system and method that facilitates the simultaneous performance of ophthalmic electrophysiology tests, pupillometry tests, and/or psychophysical tests using a single apparatus.

### BACKGROUND OF THE INVENTION

Ophthalmic electrophysiology diagnostic equipment, such as that manufactured and sold by Diagnosys LLC of Lowell, MA is typically used to stimulate the eye of a test subject using flashes, static backgrounds and/or moving patterns of light, and then to measure the resulting electrical response generated at the retina of the test subject (i.e., in order to generate an electroretinogram, or “ERG”). Additionally, such ophthalmic electrophysiology diagnostic equipment may be used to stimulate the eye of a test subject and, by using electrodes applied on or near the eye, obtain and measure the electrical response generated at the visual cortex (i.e., to obtain a visual evoked potential, or “VEP”) using electrodes applied on or near the visual cortex. To that end, electrodes (sometimes referred to as “reference” electrodes and “ground” electrodes) may be applied to other locations of the test subject's body in order to make electrical measurements that are important for performing the electroretinography test that the clinician seeks to perform.

Ophthalmic electrophysiology is considered to be the only objective measure of visual function; all other ophthalmic diagnostics are either subjective measures of visual function or a measure of anatomical structure. Ophthalmic electrophysiology generally involves stimulating the eye of a test subject with light, and then measuring the resultant electrical responses from the body of the test subject (e.g., either from the retina of the test subject, or the visual cortex of the test subject). The stimulating light may either consist of homogenous light delivered to the entire retina by a full-field stimulator (e.g., a Ganzfeld stimulator) or the stimulating light may consist of a spot or pattern of light delivered to the eye(s) of the test subject by a so-called “free-viewing screen” (e.g., a computer monitor).

Pupillometry recordings generally consist of measurements of the pupil position, shape and size obtained during static and transient presentations of light to the eye of the test subject. Static background luminance, which may be uniform or patterned, having an arbitrary luminance and color, may be used in addition to stimulating flashes of light. Alternatively, and/or additionally, rotating patterns of light (having arbitrary luminance and color) may be presented with or without the static background luminance.

Ophthalmic psychophysical diagnostic equipment stimulates the eye (or eyes) of a test subject using flashes of light, and the test subject responds by indicating whether or not they have perceived (i.e., seen) the flash(es) of light. The response from the test subject can be given verbally (e.g., “yes” or “no” or “no response”) and entered into the system by the clinician, or the test subject can register a response via an input device, e.g., a keyboard or a subject response box (sometimes referred to as a “button box”). Two exemplary ophthalmic psychophysical tests are the Full-field Stimulus Threshold (FST) and Dark Adaptometry tests.

The term “dichoptic” refers to the viewing of a separate and independent field by each eye. In dichoptic presentation, a stimulus A is presented to a first eye (e.g., the left eye) and a different stimulus B is presented to the second eye (e.g., the right eye). Dichoptic stimulators and systems typically utilize two stimulators (e.g., a flash stimulator and/or pattern stimulator); i.e., one stimulator for each eye of the test subject, which stimulator can be independently controlled in order to present the same (or different) background light, flashes and pulses of arbitrary color and luminance.

A wide array of devices have been created within each of these categories: ophthalmic electrophysiology systems, pupillometry systems, ophthalmic psychophysical test systems and dichoptic systems. Given the clinical/research importance of each ophthalmic test, and the combined value of all such tests, there are significant clinical and research benefits that would be enabled by a single device that is configured to perform any or all of these tests simultaneously.

Thus there exists a need for a single system that enables all of the following functionality in one device under one control: an ophthalmic dichoptic stimulating and dichoptic pupil recording electrophysiology, pupillometry, and psychophysical testing system.

### SUMMARY OF THE INVENTION

The present invention comprises the provision and use of a novel integrated system that enables the simultaneous (or non-simultaneous) performance of any one of the following ophthalmic tests: ophthalmic dichoptic stimulating and dichoptic pupil recording electrophysiology, pupillometry, and psychophysical testing.

In one preferred form of the present invention, there is provided a system for obtaining ophthalmic electrophysiological, pupillometry and psychophysical responses from a test subject, said system comprising:
a dichoptic stimulator for delivering a visual stimulus to the left eye of the test subject and a visual stimulus to the right eye of the test subject so as to evoke an electrophysiological response in the test subject, wherein the dichoptic stimulator comprises a left eye stimulator for delivering the visual stimulus to the left eye and a right eye stimulator for delivering the visual stimulus to the right eye;
a plurality of electrodes for receiving electrophysiological signals from the test subject;
an amplifier for amplifying electrical signals received from the plurality of electrodes;
a left eye camera for obtaining images of the left eye of the test subject, and a right eye camera for obtaining images of the right eye of the test subject;
an indicator box for inputting a psychophysical response to at least one of the visual stimulus to the left eye and the visual stimulus to the right eye;
a controller for controlling operation of at least one of the dichoptic stimulator, the left eye camera, the right eye camera and the indicator box;
a computer for processing (i) electrical signals from the amplifier, (ii) images from the left eye camera and the right eye camera, and (iii) response signals from the indicator box so as to obtain the ophthalmic electrophysiological, pupillometry and psychophysical results of the test subject.

In another preferred form of the present invention, there is provided a method for obtaining ophthalmic electrophysiological, pupillometry and psychophysical responses from a test subject, said method comprising:
providing a system for obtaining ophthalmic electrophysiological, pupillometry and psychophysical responses from a test subject, said system comprising:
   a dichoptic stimulator for delivering a visual stimulus to the left eye of the test subject and a visual stimulus to the right eye of the test subject so as to evoke an electrophysiological response in the test subject, wherein the dichoptic stimulator comprises a left eye stimulator for delivering the visual stimulus to the left eye and a right eye stimulator for delivering the visual stimulus to the right eye;
   a plurality of electrodes for receiving electrophysiological signals from the test subject;
   an amplifier for amplifying electrical signals from the plurality of electrodes
   a left eye camera for obtaining images of the left eye of the test subject, and a right eye camera for obtaining images of the right eye of the test subject;
   an indicator box for inputting a psychophysical response to at least one of the visual stimulus to the left eye and the visual stimulus to the right eye;
   a controller for controlling operation of at least one of the dichoptic stimulator, the left eye camera, the right eye camera and the indicator box;
   a computer for processing (i) electrical signals from the amplifier, (ii) images from the left eye camera and the right eye camera, and (iii) response signals from the indicator box so as to obtain ophthalmic electrophysiological, pupillometry and psychophysical results of the test subject;
using at least one of said left eye stimulator and said right eye stimulator to deliver at least one visual stimulus to at least one of the left eye of the test subject and the right eye of the test subject over a predetermined period of time;
obtaining an electrical signal representative of the electrical potential present in the at least one eye of the test subject exposed to said at least one visual stimulus;
amplifying said electrical signal using said amplifier;
obtaining at least one of an image of the left eye from the left eye camera and an image of the right eye from the right eye camera;
obtaining a response signal from the indicator box; and
processing the electrical signal from the amplifier, at least one image from the left eye camera and the right eye camera, and the response signal from the indicator box so as to obtain ophthalmic electrophysiological, pupillometry and psychophysical results of the test subject.

In another preferred form of the present invention, there is provided a method for performing pupillometry on an eye of a test subject, said method comprising:
delivering an optical stimulus to an eye of the test subject;
obtaining images of the eye of the test subject;
processing the images of the eye of the test subject, wherein processing the images comprises:
   extracting an image of the eye of the test subject;
   converting said image to a grey scale image;
   cropping said grey scale image so as to produce a cropped image;
   applying a Gaussian filter to said cropped image so as to generate a reduced noise image;
   applying a Canny edge detection algorithm to said reduced noise image so as to generate an edge image in which edges of differently-shaded anatomical structures are highlighted; and
   using a morphological transform to thicken the edges of the anatomical structures appearing in said edge image, whereby to generate a modified edge image.

In another preferred form of the present invention, there is provided apparatus for obtaining ophthalmic electrophysiological, pupillometry and psychophysical responses from a test subject, the apparatus comprising:
a housing comprising a curved surface;
a dichoptic stimulator mounted to the curved surface of the housing, the dichoptic stimulator comprising a left eye stimulator for delivering a visual stimulus to the left eye of the test subject and a right eye stimulator for delivering a visual stimulus to the right eye of the test subject, whereby to evoke an electrophysiological response in the test subject;
a pair of cameras mounted to the curved surface of the housing, wherein the pair of cameras comprises a left eye camera for obtaining images of the left eye and a right eye camera for obtaining images of the right eye of the test subject; and
an indicator box for recording a psychophysical response to at least one of the visual stimulus to the left eye and the visual stimulus to the right eye.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of a novel system for providing optical stimuli to a test subject, whereby to facilitate dichoptic electrophysiology, pupillometry and/or psychophysical response testing, formed in accordance with the present invention;

FIG. 2 is a schematic view showing further aspects of how the plurality of electrodes of the novel system depicted in FIG. 1 attach to the body of an exemplary human test subject during an ERG test;

FIGS. 3 and 4 are schematic views showing further aspects of a dichoptic stimulator housing formed in accordance with the present invention;

FIG. 5 is a schematic top view of the dichoptic stimulator housing shown in FIGS. 1-4;

FIG. 6 is a schematic view showing another novel system for providing optical stimuli to a test subject, whereby to facilitate dichoptic electrophysiology, pupillometry and/or psychophysical response testing, formed in accordance with the present invention;

FIG. 7 is a schematic view showing further aspects of how the plurality of electrodes of the novel system depicted in FIG. 6 attach to the body of an exemplary human test subject;

FIG. 8 is a schematic view showing how the novel system of FIG. 1 may be used to generate an image of the pupil of a test subject in accordance with a novel method according to the present invention;

FIG. 9 is a flow chart depicting the steps of a novel method for performing pupillometry in accordance with the present invention;

FIG. 10 is a graph showing exemplary pupillometry recordings obtained using the novel method of FIG. 9; and

FIG. 11 is a flow chart depicting the steps of a novel method for tracking eyelid movement in accordance with the present invention.

### DETAILED DESCRIPTION OF THE PREFERRED EMBODIMENTS

The present invention comprises the provision and use of a new and improved system for performing ophthalmic dichoptic stimulating and dichoptic pupil recording electrophysiology, pupillometry, and psychophysical testing, either independently, or in concert, using a system integrated into a single housing, the system comprising one controller, which can be mounted on a tabletop stand, mounted on an arm and positioned in front of a test subject, provided as a handheld device, or configured as a head-mounted device for donning by a test subject.

### 1. System Configured for Ophthalmic Dichoptic Stimulating and Dichoptic Pupil Recording Electrophysiology, Pupillometry, and Psychophysical Testing

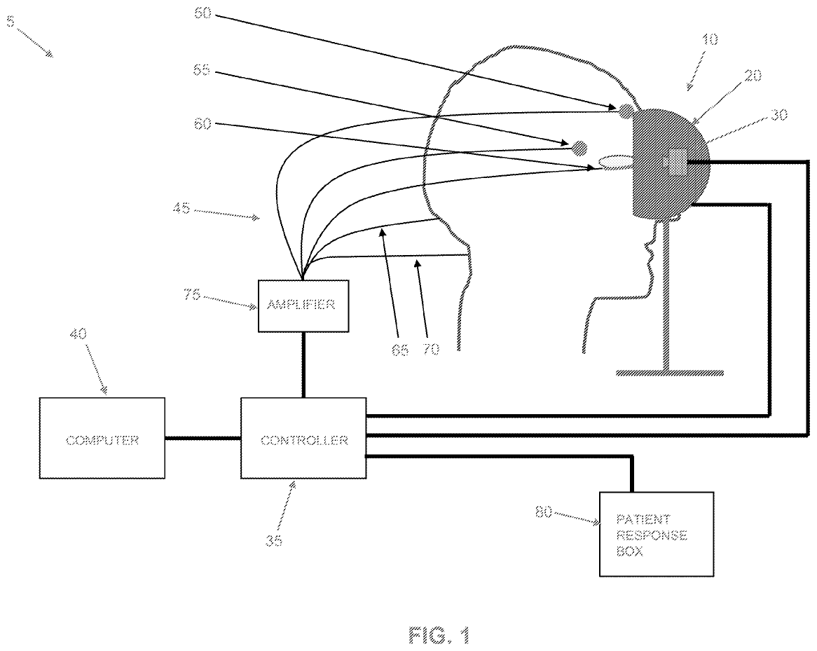

FIG. 1 is a schematic view showing a novel ophthalmic system 5. System 5 is preferably configured to provide dichoptic stimulating and dichoptic pupil recording electrophysiology, pupillometry, and psychophysical testing.

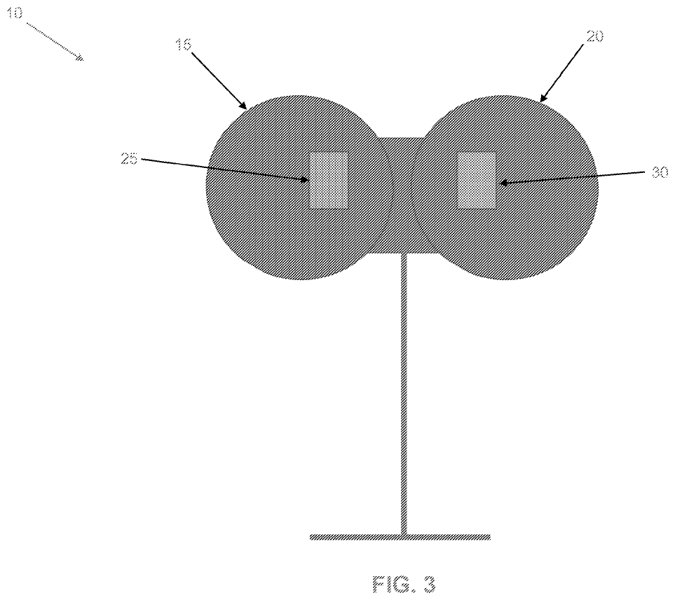

More particularly, and looking now at FIGS. 1 and 3-5, system 5 generally comprises a dichoptic stimulator housing 10 comprising a left eye stimulator 15, a right eye stimulator 20, a left eye camera 25 and a right eye camera 30. Simulators 15, 20 and cameras 25, 30 are electrically connected to a controller 35 and computer 40 configured to control operation of stimulator 10, as will hereinafter be discussed in further detail.

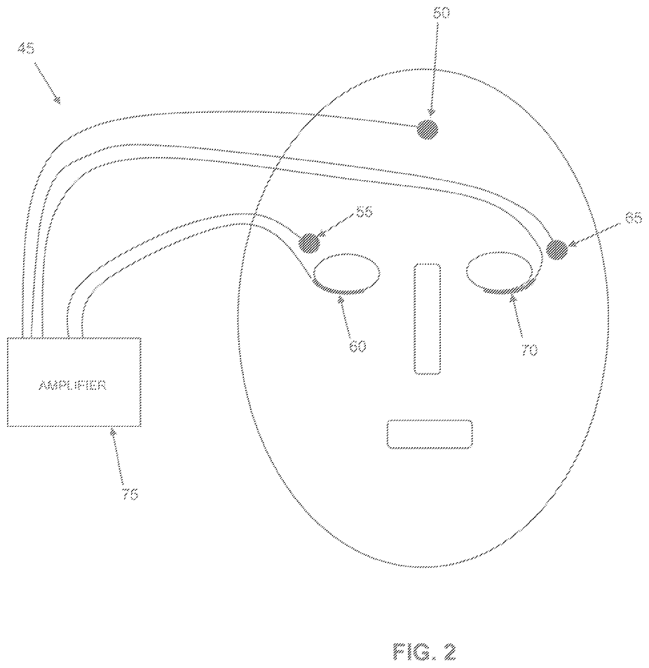

Looking now at FIGS. 1 and 2, a plurality of electrodes 45 is provided for recording electrical responses from the eye(s) of the test subject. In one preferred form of the invention, plurality of electrodes 45 comprises a ground electrode 50 for contacting the skin of the test subject at a location away from the eye(s) of the test subject (e.g., the center of the forehead), a right eye reference electrode 55 for contacting the skin of the test subject proximate to the right eye of the test subject, a right eye active electrode 60 for contacting the right eye of the test subject, a left eye reference electrode 65 for contacting the skin of the test subject proximate to the left eye of the test subject, and a left eye active electrode 70 for contacting the left eye of the test subject. Electrodes 50, 55, 60, 65, 70 are electrically connected to an amplifier 75 for amplifying electrical signals obtained from the plurality of electrodes 45. Amplifier 75 is, in turn, electrically connected to controller 35 (and hence, in turn, to computer 40).

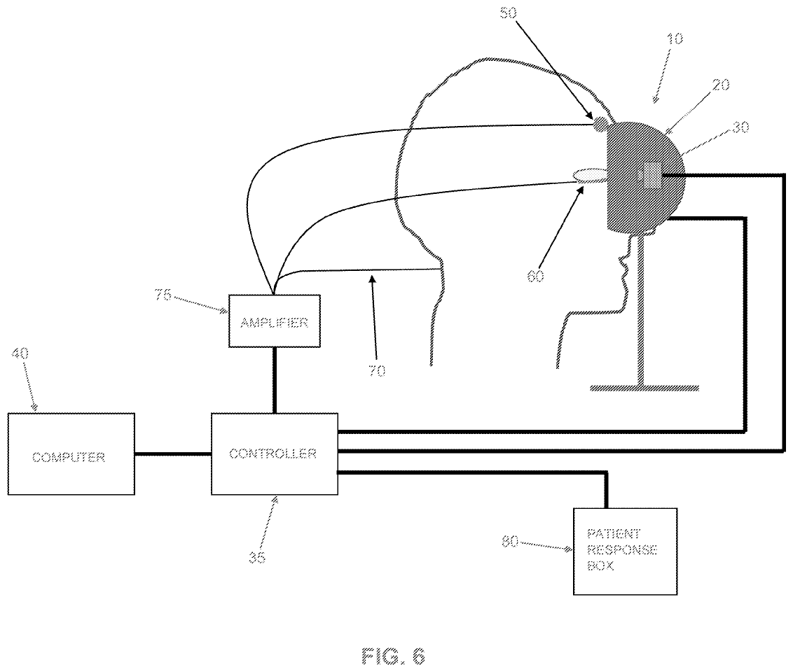

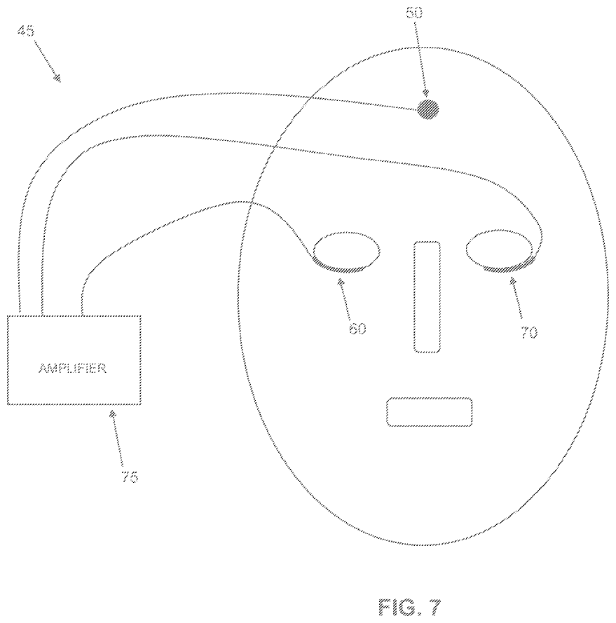

It should be appreciated that, if desired, and looking now at FIGS. 6 and 7, reference electrodes 55, 65 may be omitted, and each of the test subject's eyes may be tested individually (i.e., such that one eye is stimulated while the fellow eye is not stimulated). With this form of the invention, the active electrodes 60, 70 in contact with the unstimulated fellow eye is used as a reference electrode while the other eye is being stimulated, and vice-versa.

All aspects of the system 5 (i.e., stimulators 15, 20; cameras 25, 30; electrodes 50, 55, 60, 65, 70; amplifier 75, etc.) are under one common control (i.e., controller 35). Amplifier 75 is preferably a multi-port amplifier, and amplifier 75, controller 35 and computer 40 are configured for recording ERG, visual evoked potential (VEP) or electro-oculogram (EOG) signals. Pupillometry hardware, firmware and software are provided (e.g., running on computer 40) to record pupil diameter and area of one or both eye(s) of the test subject before, during and after either (or both) of the test subject's eyes are stimulated by left eye stimulator 15 and/or right eye stimulator 20, which pupil diameter and area is obtained by either (or both) cameras 25, 30 at any moment in time. A patient response box 80 (e.g., a button push, a keyboard push, a verbal trigger, a hand motion trigger, etc.) is provided in order to facilitate psychophysical testing by capturing “yes” or “yes/no” responses when prompted or instructed to register a response using patient response box 80 in response to delivery of a light stimulus via stimulators 15, 20. A computer and software program running on computer 40 provides an interface for the clinician performing the test, recording, analyzing and storing the test data. Computer 40 may be a separate component from the other components of system 5 (e.g., a laptop computer, a desktop computer, a tablet computer, etc.) or computer 40 may be integrated together with the other components (i.e., dichoptic stimulator housing 10; stimulators 15, 20; cameras 25, 30; controller 35; plurality of electrodes 45; amplifier 75; and patient response box) of system 5.

System 5 enables ophthalmic electrophysiology, pupillometry, and psychophysical testing to be performed using one system and for the tests to be combined. Numerous unique combinations of ophthalmic tests are made possible by system 5. By way of example but not limitation, such combinations include recording an electrophysiology signal (ERG and/or VEP) simultaneously with the acquisition of a pupillometry trace, while also simultaneously conducting a psychophysical test, with all of the ophthalmic tests being performed relying on the same stimulus (or stimuli).

By way of example but not limitation, such “combined” testing can be performed by stimulating both eyes of the test subject with the same light stimulus (e.g., on the same background light, or no background light) or by using different light stimuli for each eye, or by stimulating only one eye of the test subject at any given time. It should be appreciated that any subset of these ophthalmic tests can be conducted at the same time during the same stimulus (or set of stimuli). Significantly, all ophthalmic tests (or any subset) can be interleaved with each other in an arbitrary combination and ordering of tests for each and both eyes of the test subject.

### 2. Method for Pupillometry Analysis

In addition to the provision of a comprehensive system that permits simultaneous performance of tests relating to ophthalmic electrophysiology, pupillometry, and psychophysical testing, it should be appreciated that the present invention also comprises a novel method for performing pupillometry analysis.

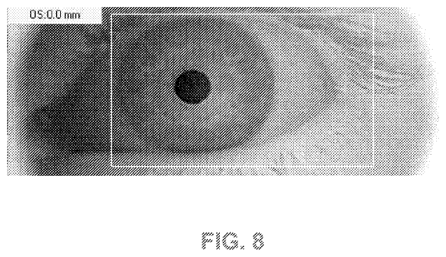

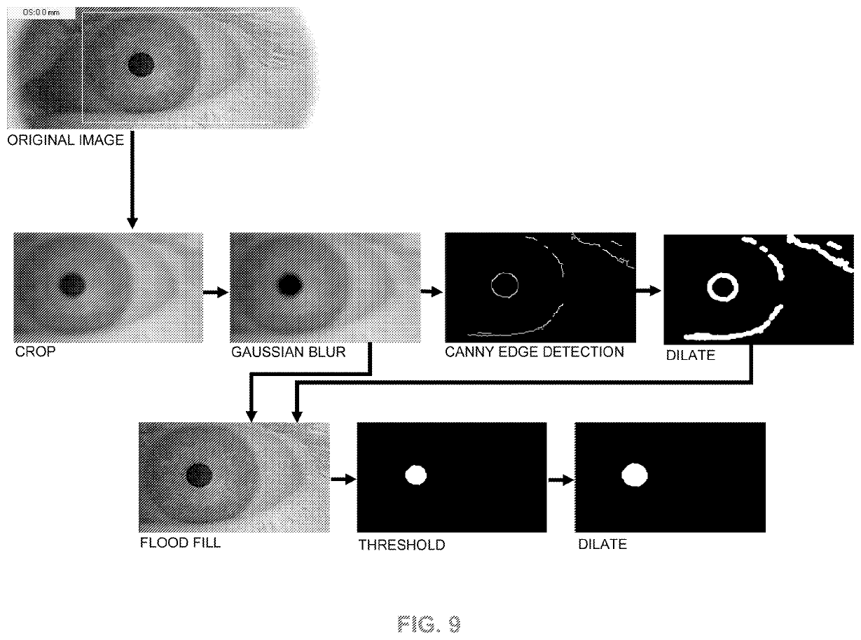

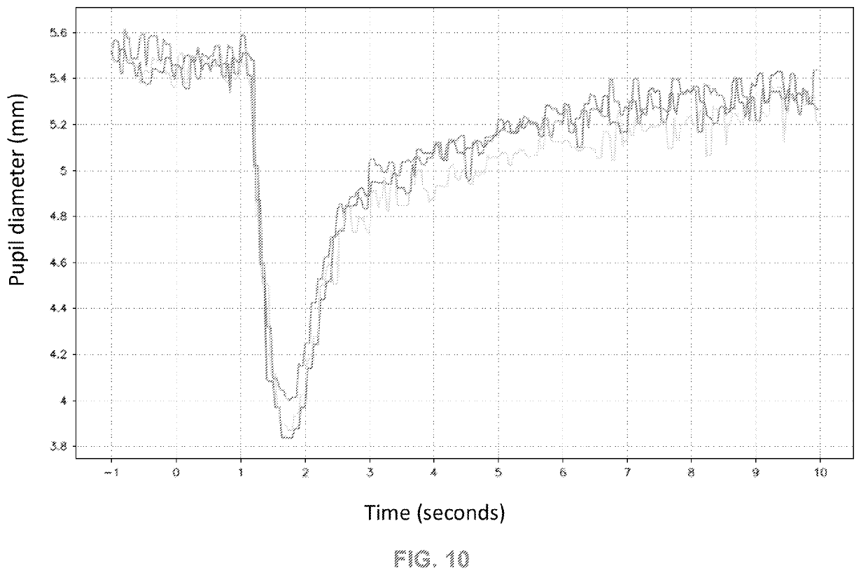

More particularly, and looking now at FIGS. 8 and 9, the present invention comprises a new, highly accurate pupillometry analysis system and method.

According to the novel method of the present invention, a digital or analog camera (e.g., the aforementioned left eye camera 25 and/or the aforementioned right eye camera 30) records an image of the eye of the test subject in order to monitor the response of the eye to a stimulus. The video stream produced by the camera (e.g., one or both of cameras 25, 30) is then processed in software (e.g., software running on the aforementioned computer 40), whereby to measure the location and size of the pupil in each frame of video. See FIG. 8.

Looking now at FIG. 9, the processing of each video frame (i.e., image) proceeds in several stages:

1. First, if the image is in color, the image is converted to grey scale.

2. The image is also cropped to focus on a region of interest which contains the pupil. Performing all subsequent processing on only the region of interest instead of the entire video frame increases processing speed of the image.

3. Next a Gaussian filter is applied to the cropped image, whereby to create a blurred image in order to reduce noise.

4. A Canny edge detection algorithm is then used to produce a second “edge image” which highlights edges of differently-shaded structures in the image. The Canny edge detection algorithm produces a binary image with all edges in the image highlighted. These edges include the boundary of the pupil. However, the boundary is not always perfectly defined, and may contain gaps. Furthermore, in addition to the pupil boundary of interest, various other edges corresponding to anatomical structures not relevant to pupillometry analysis are normally present in this image.

5. The edge image is then dilated using a morphological transform. This function thickens all the edges identified by the Canny edge detection algorithm and, in the process, closes any “gaps” which may be present in the pupil boundary.

6. The software then locates the pixel in the blurred image derived in Step 3 having the minimum brightness, which pixel is generally a pixel located within the pupil (inasmuch as the region of the pupil is generally darker than the surrounding region of the eye).

7. Next a “flood fill operation” is performed on the blurred image starting at the pixel derived in Step 6 (i.e., the pixel having minimum brightness), whereby to fill the pupil region abutting the minimum brightness pixel with black. During the flood fill operation, the dilated boundary image derived in Steps 4 and 5 is used as a mask to prevent the flood fill operation from filling any regions outside the pupil.

8. A threshold is then used to generate a binary image corresponding to the region which was filled black. This region represents the pupil up to the dilated edge mask.

9. To compensate for the thickness of the mask resulting from the morphological transform applied in Step 5, the binary image is then dilated by the same edge image kernel which was used in Step 5 to dilate edges. This results in a binary image which cleanly identifies the pupil, without reducing the size of the pupil by the area of thickened boundary resulting from the application of the morphological transform in Step 5.

10. The software then computes the image moments (i.e., a particular weighted average of the intensities of the pixels of the image) for the image derived in Step 9. In one preferred form of the invention, the image moments are computed using the following equation:

$${M}_{ij} = {\sum }_{x} {\sum }_{y} {x}^{i} {y}^{i} I ( x , y )$$

M₀₀ represents the area of the pupil. Pupil diameter is then calculated from this area by assuming a circular shape. Also, the location of the centroid of the pupil can be calculated from (M₁₀/M₀₀, M₀₁ M₀₀).

Alternatively, the width and height of the pupil can be measured by identifying the bounding rectangle for the region obtained in the image derived in Step 9.

11. The software (e.g., software running on the aforementioned computer 40) also computes the Hu Moments of the image derived in Step 9. Hu Moments are a set of numbers computed from the image moments of Step 10, which are translation and scale invariant. In particular the first Hu Moment can be used to verify that the region in the image derived in Step 9 is circular. Any video frames in which the region deviates from a perfect circle by too much are discarded from the analysis.

The recorded data can then be further filtered, analyzed, averaged and other post-processing methods that will be apparent to those of skill in the art in view of the present disclosure.

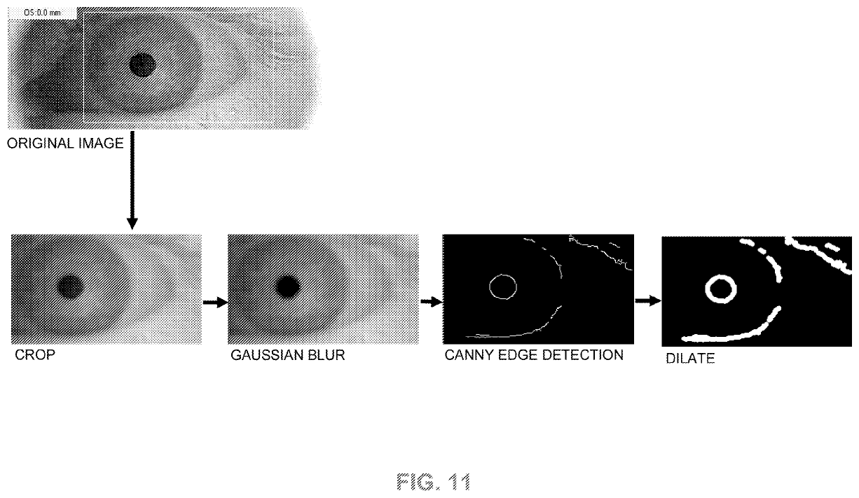

FIG. 8 shows an example of an initial video frame obtained from the eye of a test subject using one of the aforementioned cameras 25, 30. FIG. 9 depicts the image processing sequence of the novel method of the present invention (i.e., Steps 1-11 discussed above). FIG. 11 shows an example pupil diameter recording over time before, during and after delivery of a light stimulus (e.g., a pulse of light) to the eye of the test subject. The novel method of the present invention is extremely robust and can work over a full range of stimuli, including flashes of arbitrary color, length and luminance, while maintaining the ability to detect pupil diameter/area changes of 1%. This utility over a broad range of different stimuli while retaining the ability to recognize extremely small changes in pupil diameter/area represents a significant improvement over prior art methods.

While the foregoing method is discussed in the context of performing pupillometry, if desired, the novel method of the present invention may also be used to track movement of other anatomical structures. By way of example but not limitation, the novel method of the present invention may also be used to detect or track eyelid movements during the performance of ophthalmic tests.

To that end, and looking now at FIG. 11, there is shown a set of steps in the image processing sequence used to track the position of each eyelid of a test subject. In each case, only a portion (e.g., an edge) of the eye lid is tracked, and its position is tracked relative to either the other eyelid or relative to the pupil of each eye.

It will be appreciated that with the modified method of FIG. 11 used to track movement of the eyelid, it is not necessary to calculate the area of the image occupied by the eyelid itself. Thus, with this form of the invention, Steps 6-11 discussed above may be omitted. Therefore, the modified method of FIG. 11 would proceed as follows:

1. First, if the image is in color, the image is converted to grey scale.

2. The image is also cropped to focus on a region of interest which contains the eyelid that is to be tracked. Performing all subsequent processing on only the region of interest instead of the entire video frame increases processing speed of the image.

3. Next a Gaussian filter is applied to the cropped image, whereby to create a blurred image in order to reduce noise.

4. A Canny edge detection algorithm is then used to produce a second “edge image” which highlights edges of differently-shaded structures in the image. The Canny edge detection algorithm produces a binary image with all edges in the image highlighted. These edges include the boundary of the eyelid (e.g., the lower edge of the upper eyelid). However, the boundary is not always perfectly defined, and may contain gaps. Furthermore, in addition to the eyelid boundary of interest, various other edges corresponding to anatomical structures not relevant to tracking eyelid movement are normally present in this image.

5. The edge image is then dilated using a morphological transform. This function thickens all the edges identified by the Canny edge detection algorithm and, in the process, closes any “gaps” which may be present in the eyelid boundary.

### 3. Dichoptic Stimulator Housing 10 Comprising Improved Form Factor

Traditionally, ophthalmic stimulators (Ganzfelds, pattern, flash paddle that are binocular, monocular, or dichoptic) used for ophthalmic electrophysiology, pupillometry, and psychophysical tests comprise a flat front surface (e.g., because it is easier to manufacture). However, the human face is not flat. Therefore, it would be desirable to provide a dichoptic stimulator that better conforms to the natural geometry of the human face.

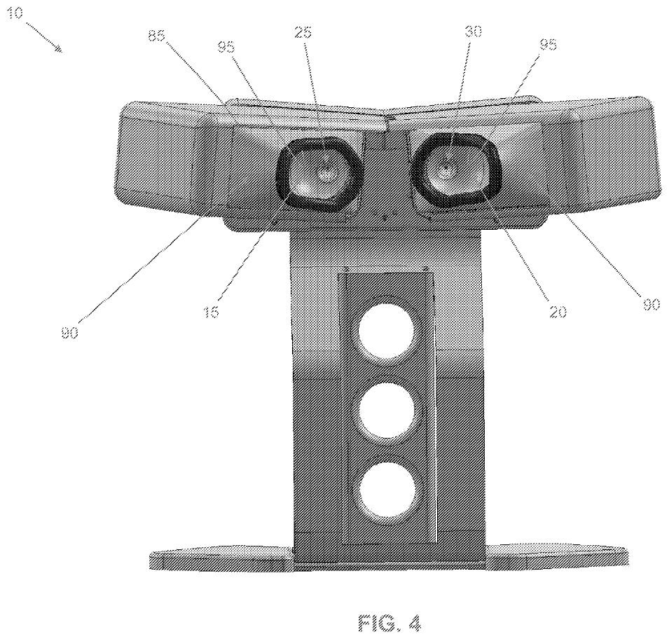

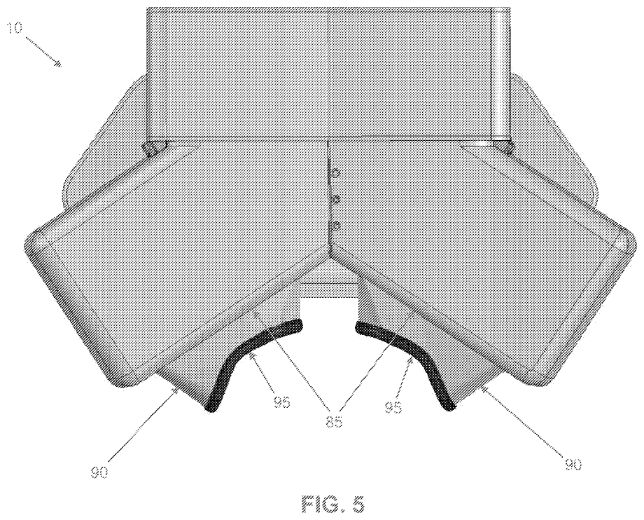

To that end, and looking again at FIGS. 4 and 5, there is shown a novel dichoptic stimulator housing 10 comprising a curved surface 85 configured to contact the face of the test subject (or reside immediately in front of the test subject's face). If desired, blinds 90 may be mounted to curved surface 85 so as to accommodate the test subject's eye(s) and create a light-seal between the test subject's eye(s) and curved surface 85 (which surface contains the aforementioned stimulators 15, 20). Blinds 90 preferably comprise a flexible, opaque material (e.g., rubber) and an opening 95 sized to fit around the perimeter of the test subject's eye(s), whereby to securely seat against the test subject's face. In this manner, the test subject's eye(s) are isolated from any ambient light, such that the only light entering the test subject's eye(s) is the stimuli provided by simulators 15, 20.

Thus, it will be appreciated that curved surface 85 provides numerous benefits, including greater test subject comfort as well as greater coverage of the test subject's field of view by the stimulus in a very compact package.

### 4. Improved Stimulator Flash and Background Range

Traditionally, flash stimulators (e.g., Ganzfelds, pattern, flash paddle that are binocular, monocular, or dichoptic) used for ophthalmic electrophysiology, pupillometry, and psychophysical tests have been limited to a dimmest 4-millisecond flash of approximately 10⁻⁹ candela*seconds/m² (and an equivalent background luminance in candela/m²). The present invention utilizes stimulators 15, 20 comprising an optical design which enables the extension of well-controlled flashes (and equivalent background luminance) well below 10⁻¹⁰ candela*seconds/m² down to as low as 10⁻¹² candela*seconds/m² and as high as 10⁺³ candela*seconds/m², a highly uniform presentation of light across the field of view of the eye. In addition, the provision of curved surface 85 of stimulator housing 10 permits cameras 25, 30 to be positioned within a distance that is less than 2 inches away from the eye.

### 5. Dichoptic Stimulation

Novel ophthalmic system 5 also enables psychophysical tests such as Dark Adaptometry and Full-field Stimulus Threshold (FST) to be performed using a dichoptic stimulator (i.e., stimulators 15, 20) configured such that each eye of the test subject can be (i) tested separately (e.g., while the non-tested eye is kept at a constant luminance or in a dark-adapted state, under controlled lighting conditions); (ii) the eyes of the test subject can be tested together using the same stimulus at the same time; or (iii) the eyes of the test subject can be stimulated independently with system 5, uniquely enabling the presentation of different stimuli to each eye during psychophysical tests.

In addition, if desired, consensual response measurements (e.g., stimulating one eye of the test subject with a flash of light stimulus, while recording the pupil response in the unstimulated, fellow eye) can be done for the first time at luminance levels down to and below the human light sensitivity threshold (which is approximately 10⁻⁷ to 10⁻⁸ candela*seconds/m² in 6500K white light and corresponding values for individual colors) in a 4 millisecond flash or in steady-state background conditions.

### 6. Monitoring of Artifacts

Twin cameras (e.g., the aforementioned cameras 25, 30) allow for simultaneous pupillometry, eye gaze tracking, and eyelid position tracking in both eyes of the test subject, whereby to enable a variety of useful functions not previously possible with prior art systems.

By way of example but not limitation, cameras 25, 30 can be used to monitor pupil response to stimuli of either the ipsilateral eye or the contralateral (i.e., fellow) eye, or both.

Cameras 25, 30 can also be used to monitor small twitches in the test subject's eyelids that might induce artifacts on the ERG response that are too subtle to detect by monitoring the electrical signal alone.

Similarly, independent eye gaze tracking using cameras 25, 30 can also be used to detect involuntary saccades, which movements can also induce electrical artifacts in the recorded ERG response.

Cameras 25, 30 can also monitor and correct for over or under excursions in electro-oculography (EOGs), helping normalize responses in patients having poor motor control who might otherwise be impossible to test.

Controller 35 (and/or computer 40) uses at least one piece of information from each of the above examples to monitor the quality of the electrophysiology or psychophysical recorded response, and then either rejects the recording made during a period of time when the artifact was too strong and/or modifies (e.g., corrects) the recorded signal from the electrophysiology or psychophysical response based on video analysis of the moving eye and/or eyelid. During these tests each eye of the test subject will react slightly differently, and each reaction causes its own artifact for the test that is being performed. Having two independent cameras 25, 30, each camera monitoring one eye simultaneously with the other camera monitoring the other eye, enables the identification of each eye's artifacts independently, and for each artifact to be corrected for independently. This is clinically very valuable to the clinician since it allows for electrophysiology and psychophysical measurements that are more dependent on the capabilities of the retina and neural vision system and less dependent on outer eye artifacts than with prior art systems, thus enabling a more direct measurement of what is of interest to the clinician.

### 7. Dichoptic Electro-Oculography (EOG)

Because the entirety of system 5, including the two stimulators 15, 20 (and their associated lighting systems, including fixation point LED lights), are under one common control, it is possible to conduct tests where synchronized control and alternative lighting of more than 1 LED fixation point light is possible. Thus it is possible, for the first time, to perform tests such as EOG on a dichoptic stimulator.

### 8. Additional Improvements

In one form of the invention, stimulator housing 10 comprises a Ganzfeld (i.e., full-field) stimulator. More particularly, in this form of the invention, the system incorporates, in a single binocular-sized stimulator housing 10, the following:
a pair of dichoptic full-field stimulators 15, 20, each configured to stimulate one eye without stimulating the other, and each stimulator 15, 20 incorporating in-focus fixation points at centerline and +/−15 degrees;
a pair of cameras 25, 30, with each camera 25, 30 being configured to observe an eye of the test subject;
a three-channel amplifier 75 (it should be appreciated that three channels are the minimum number required for performing the ophthalmic tests envisioned by the present invention, with each channel recording both an active and reference signal, however, if desired, an amplifier comprising more than three channels may be provided) to record an ERG obtained from one eye while using the other, fellow eye as a reference (e.g., by using right eye active electrode 60 to measure the electrical response of the right eye to a stimulus produced by right eye stimulator 20 while left eye active electrode 70 is used as a reference electrode);
a high-quality stereo sound system for providing information to the test subject, and possessing sufficient quality to perform audio testing on the test subject;
a built-in (e.g., integrated into stimulator 10) control screen and control electronics facilitating complete control over stimulus and acquisition functions of system 5.

Also included are USB and WiFi interfaces by means of which one or more systems can connect to a central computer for expanded user interface, data manipulation, and data storage.

Each single-eye stimulator 15, 20 preferably incorporates an array of LEDs configured to emit light at different wavelengths, which LEDs are driven together and can produce any of seven discrete wavelengths, or any metameric combination of seven wavelengths, of visible light. These wavelengths include, but are not limited to 680 nm, 630 nm, 590 nm, 525 nm, 470 nm, 450 nm, white phosphor, and infrared (camera illumination). All wavelengths except infrared are available over an extraordinary 12-order-of-magnitude range of luminance. The large selection of wavelengths is useful in isolating cone responses for assessment of various forms of hereditary eye disease.

Such a combination of capabilities enables a wide variety of applications not possible with prior art Ganzfeld stimulators.

Because the new Ganzfeld systems (i.e., stimulators 15, 20) of the present invention are much brighter than prior art Ganzfelds, stimulators 15, 20 of the present invention can be used in intensity series electrophysiology measurements that extend beyond the range of prior art Ganzfeld stimulators. Because the stimulus can be applied to one eye only, a very bright stimulus cause less “flinch artifact” than if applied to both eyes simultaneously, further extending its utility.

For the same reason, novel ophthalmic system 5 can be used in psychophysical testing such as dark adaptometry and full-field stimulus threshold (FST) testing, even when performed on a test subject possessing very limited light perception. This makes system 5 ideal for the development and patient screening involved in the burgeoning new class of genetic treatments for eye diseases.

Novel ophthalmic system 5 is configured to perform at least the following tests, sequentially or in combination: Photopic and scotopic full-field flash ERG, flicker ERG, photopic negative ERG, s-cone ERG, red-flash scotopic ERG, flash VEP (three, two, or one channel; the last simultaneously or without ERG), EOG (under dual camera observation to correct response amplitudes for erratic excursions), full-field stimulus threshold, dichoptic pupillometry, and gaze tracking, the latter two useful in diagnosing traumatic brain injuries (TBIs) and neurological disorders. Audio stimuli, including click and shaped sinusoidal tones are also useful in conjunction with pupillometry and gaze-tracking for diagnosing TBI and neurological disorders.

Through the utilization of time domain multiplexing, the completely independent control of background and flash, across the full range of color and light intensity, delivered by stimulators 15, 20 to both eyes of the test subject permits a much broader range of tests than otherwise possible, and makes possible a simpler and much less noisy method of data acquisition when using the fellow non-stimulated eye as the reference for the active recording on the stimulated eye. System 5 also enables dichoptic VEP presentations useful in assessing binocular vision in infants, and dichoptic psychophysical testing, in which different adapting backgrounds and stimulus conditions can be simultaneously presented to both eyes, which may be useful in detecting defects in pupil response pathways.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A system for obtaining ophthalmic electrophysiological, pupillometry and psychophysical responses from a test subject, said system comprising:
a dichoptic stimulator for delivering a visual stimulus to the left eye of the test subject and a visual stimulus to the right eye of the test subject so as to evoke an electrophysiological response in the test subject, wherein the dichoptic stimulator comprises a left eye stimulator for delivering the visual stimulus to the left eye and a right eye stimulator for delivering the visual stimulus to the right eye;
a plurality of electrodes for receiving electrophysiological signals from the test subject;
an amplifier for amplifying electrical signals received from the plurality of electrodes;
a left eye camera for obtaining images of the left eye of the test subject, and a right eye camera for obtaining images of the right eye of the test subject;
an indicator box for inputting a psychophysical response to at least one of the visual stimulus to the left eye and the visual stimulus to the right eye;
a controller for controlling operation of at least one of the dichoptic stimulator, the left eye camera, the right eye camera and the indicator box;
a computer for processing (i) electrical signals from the amplifier, (ii) images from the left eye camera and the right eye camera, and (iii) response signals from the indicator box so as to obtain the ophthalmic electrophysiological, pupillometry and psychophysical results of the test subject.

2. The system of claim 1 wherein the plurality of electrodes comprise a left eye active electrode in contact with the left eye of the test subject, a right eye active electrode in contact with the right eye of the test subject and a ground electrode in contact with the skin of the test subject.

3. The system of claim 2 wherein the plurality of electrodes further comprise a left eye reference electrode in contact with the test subject proximate to the left eye of a test subject and a right eye reference electrode in contact with the test subject proximate to the right eye of the test subject.

4. The system of claim 1 wherein said right eye stimulator and said right eye camera are optically isolated from said left eye stimulator and said left eye camera.

5. The system of claim 1 wherein said right eye stimulator, said right eye camera, said left eye stimulator and said left eye camera are mounted to a curved surface approximating the curvature of a human face.

6. The system of claim 1 wherein said right eye stimulator and said left eye stimulator are configured to deliver stimuli to the left eye and the right eye independently of one another at different times.

7. The system of claim 6 wherein one of said plurality of electrodes measures the electrical potential from the eye being stimulated and generates a corresponding electrical signal and another one of said plurality of electrodes measures the electrical potential from the eye not being stimulated and generates a corresponding electrical signal, whereby to serve as a reference electrode.

8. The system of claim 1 wherein said right eye stimulator and said left eye stimulator are configured to simultaneously deliver stimuli independently of one another.

9. The system of claim 8 wherein said stimulus delivered by said right eye stimulator differs from said stimulus delivered by said left eye stimulator.

10. The system of claim 8 wherein:
when said right eye stimulator delivers the visual stimulus to the right eye of the test subject, one of the plurality of electrodes measures the electrical potential in the right eye of the test subject and generates a corresponding right eye electrical signal;
when said left eye stimulator delivers the visual stimulus to the left eye of the test subject, a different one of the plurality of electrodes measures the electrical potential in the left eye of the test subject and generates a corresponding left eye electrical signal; and
wherein said computer is configured to produce a graphical representation of the variation of said right eye electrical signal and said left eye electrical signal over a predetermined period of time, whereby to generate an elecro-oculogram (EOG) for the right eye of the test subject and for the left eye of the test subject.

11. The system of claim 1 wherein said computer uses the images from said left eye camera and said right eye camera to calculate the size of the pupil of the left eye of the test subject and the size of the pupil of the right eye of the test subject.

12. The system of claim 11 wherein the size comprises at least one of diameter of the pupil and area of the pupil.

13. The system of claim 11 wherein said computer is configured to (i) record the size of the pupil of the left eye of the test subject and the size of the pupil of the right eye of the test subject over a predetermined period of time, and (ii) produce a graphical representation of the variation of pupil size over said predetermined period of time.

14. The system of claim 13 wherein the predetermined period of time starts before the visual stimulus is delivered to at least one of the left eye and the right eye and continues until a point in time after the visual stimulus has been delivered to at least one of the left eye and the right eye.

15. The system of claim 1 wherein said computer uses the images from said left eye camera and said right eye camera to determine the location of at least one of the left eyelid and the right eyelid of the test subject.

16. The system of claim 15 wherein said computer is configured to record the location of the left eyelid of the test subject and/or the location of the right eyelid of the test subject over a predetermined period of time and to produce a graphical representation of the movement of the left eyelid and the right eyelid over said predetermined period of time.

17. The system of claim 16 wherein the predetermined period of time starts before the visual stimulus is delivered to at least one of the left eye and the right eye and continues until a point in time after the visual stimulus has been delivered to at least one of the left eye and the right eye.

18. The system of claim 1 wherein the psychophysical response comprises an indication that the test subject perceived at least one of the visual stimulus delivered to the left eye and the visual stimulus delivered to the right eye.

19. The system of claim 18 wherein said computer is configured to record said response signal from said indicator box and generate a graphical representation of said response signal against said delivery of the visual stimulus to the left eye and the visual stimulus to the right eye.

20. The system of claim 19 wherein at least one of said visual stimulus delivered to the left eye of the test subject and said visual stimulus delivered to the right eye of the test subject is selected so as to perform a Full-field Stimulus Threshold Test (FST) on the test subject, and further wherein said graphical representation comprises the results of said Full-field Stimulus Threshold Test (FST).

21. The system of claim 19 wherein at least one of said visual stimulus delivered to the left eye of the test subject and said visual stimulus delivered to the right eye of the test subject is selected so as to perform a Dark Adaptometry Test on the test subject, and further wherein said graphical representation comprises the results of said Dark Adaptometry Test.

22. A method for obtaining ophthalmic electrophysiological, pupillometry and psychophysical responses from a test subject, said method comprising:
providing a system for obtaining ophthalmic electrophysiological, pupillometry and psychophysical responses from a test subject, said system comprising:
a dichoptic stimulator for delivering a visual stimulus to the left eye of the test subject and a visual stimulus to the right eye of the test subject so as to evoke an electrophysiological response in the test subject, wherein the dichoptic stimulator comprises a left eye stimulator for delivering the visual stimulus to the left eye and a right eye stimulator for delivering the visual stimulus to the right eye;
a plurality of electrodes for receiving electrophysiological signals from the test subject;
an amplifier for amplifying electrical signals from the plurality of electrodes
a left eye camera for obtaining images of the left eye of the test subject, and a right eye camera for obtaining images of the right eye of the test subject;
an indicator box for inputting a psychophysical response to at least one of the visual stimulus to the left eye and the visual stimulus to the right eye;
a controller for controlling operation of at least one of the dichoptic stimulator, the left eye camera, the right eye camera and the indicator box;
a computer for processing (i) electrical signals from the amplifier, (ii) images from the left eye camera and the right eye camera, and (iii) response signals from the indicator box so as to obtain ophthalmic electrophysiological, pupillometry and psychophysical results of the test subject;
using at least one of said left eye stimulator and said right eye stimulator to deliver at least one visual stimulus to at least one of the left eye of the test subject and the right eye of the test subject over a predetermined period of time;
obtaining an electrical signal representative of the electrical potential present in the at least one eye of the test subject exposed to said at least one visual stimulus;
amplifying said electrical signal using said amplifier;
obtaining at least one of an image of the left eye from the left eye camera and an image of the right eye from the right eye camera;
obtaining a response signal from the indicator box; and
processing the electrical signal from the amplifier, at least one image from the left eye camera and the right eye camera, and the response signal from the indicator box so as to obtain ophthalmic electrophysiological, pupillometry and psychophysical results of the test subject.

23. The method of claim 22 wherein the visual stimulus is delivered simultaneously with obtaining images of the left eye and the right eye and obtaining the psychophysical response to the visual stimulus.

24. The method of claim 23 further comprising generating a graphical representation of the electrophysiological, pupillometry and psychophysical results over time.

25. The method of claim 22 wherein both the left eye and the right eye are stimulated simultaneously using the same visual stimulus.

26. The method of claim 22 wherein both the left eye and the right eye are stimulated simultaneously using a different visual stimulus.

27. The method of claim 22 wherein the left eye and the right are stimulated at different times.

28. A method for performing pupillometry on an eye of a test subject, said method comprising:
delivering an optical stimulus to an eye of the test subject;
obtaining images of the eye of the test subject;
processing the images of the eye of the test subject, wherein processing the images comprises:
extracting an image of the eye of the test subject;
converting said image to a grey scale image;
cropping said grey scale image so as to produce a cropped image;
applying a Gaussian filter to said cropped image so as to generate a reduced noise image;
applying a Canny edge detection algorithm to said reduced noise image so as to generate an edge image in which edges of differently-shaded anatomical structures are highlighted; and
using a morphological transform to thicken the edges of the anatomical structures appearing in said edge image, whereby to generate a modified edge image.

29. The method of claim 28 wherein processing the images further comprises:
locating a pixel in said reduced noise image having minimum brightness;
using said modified edge image as a mask so as to demarcate the outer boundary of the region of interest on said reduced noise image;
applying a flood fill operation to said reduced noise image, whereby to fill in the area abutting said pixel having minimum brightness with black up to said outer boundary;
generating a binary image corresponding to said region of interest filled with black; and
calculating the area of said region of interest filled with black.

30. The method of claim 22 further comprising:
dilating the region of interest in said binary image by the same edge image kernel applied by said morphological transform to generate said modified edge image, whereby to remove said thickened edges from said binary image; and
calculating the area of said region of interest filled with black.

31. The method of claim 22 wherein the method further comprises calculating at least one from the group consisting of pupil diameter, location of the centroid of the pupil, width of the pupil and height of the pupil.

32. The method of claim 31 wherein the method further comprises plotting changes in the diameter of the pupil in response to different optical stimuli over a predetermined period of time.

33. The method of claim 28 wherein the method further comprises tracking movement of an anatomical structure while delivering the optical stimulus to the test patient over a predetermined period of time.

34. The method of claim 33 wherein the anatomical structure is an eyelid of the test subject.

35. Apparatus for obtaining ophthalmic electrophysiological, pupillometry and psychophysical responses from a test subject, the apparatus comprising:
a housing comprising a curved surface;
a dichoptic stimulator mounted to the curved surface of the housing, the dichoptic stimulator comprising a left eye stimulator for delivering a visual stimulus to the left eye of the test subject and a right eye stimulator for delivering a visual stimulus to the right eye of the test subject, whereby to evoke an electrophysiological response in the test subject;
a pair of cameras mounted to the curved surface of the housing, wherein the pair of cameras comprises a left eye camera for obtaining images of the left eye and a right eye camera for obtaining images of the right eye of the test subject; and
an indicator box for recording a psychophysical response to at least one of the visual stimulus to the left eye and the visual stimulus to the right eye.

36. The apparatus of claim 35 further comprising a plurality of electrodes for recording electrophysiological signals of the test subject and an amplifier for amplifying electrical signals received from the plurality of electrodes.

37. The apparatus of claim 35 further comprising a controller for controlling operation of the left eye stimulator, the right eye stimulator, the left eye camera, the right eye camera and the indicator box.

38. The apparatus of claim 35 further comprising a computer for processing (i) electrical signals from the test subject, (ii) images from said left eye camera and said right eye camera, and (iii) response signals from said indicator box so as to obtain ophthalmic electrophysiological, pupillometry and psychophysical results of the test subject.

39. The apparatus of claim 35 wherein the apparatus is configured to stimulate one eye of the test subject while recording a pupil response in an unstimulated eye of the test subject.

40. The apparatus of claim 35 wherein the pair of cameras simultaneously obtain images of the left eye of the test subject and the right eye of the test subject.

41. The apparatus of claim 40 wherein the images obtained from the left eye of the test subject and the right eye of the test subject are processed to track at least one of pupil diameter, eye gaze direction and eyelid position.

42. The apparatus of claim 41 wherein at least one of pupil diameter, eye gaze direction and eyelid position is used to confirm the quality of at least one of the electrophysiological response and the psychophysical response.

43. The apparatus of claim 41 wherein at least one of pupil diameter, eye gaze direction and eyelid position is used to modify at least one of the electrophysiological response and the psychophysical response.
